(19) United States
(12) Patent Application Publication
(10) Pub. No.: US 2023/0382884 A1
(43) Pub. Date: Nov. 30, 2023

(54) METHODS FOR SEPARATION AND ISOLATION OF TETRAHYDROCANNABIVARIN

(71) Applicant: Schwazze Biosciences, LLC, Denver, CO (US)

(72) Inventors: James Edward Parco, Palmer Lake, CO (US); Remy Pierre Kachadourian, Denver, CO (US)

(21) Appl. No.: 18/324,250

(22) Filed: May 26, 2023

Related U.S. Application Data
(60) Provisional application No. 63/347,542, filed on May 31, 2022.

(51) Int. Cl. C07D 311/80; B01D 15/16; B01D 1/22; B01D 15/32; B01D 15/42; B01D 5/00

(52) U.S. Cl. CPC C07D 311/80; B01D 15/165; B01D 1/225; B01D 15/325; B01D 15/426; B01D 5/006

(57) ABSTRACT

Provided herein are methods for the isolation of THCV from a composition comprising THCV and at least one other cannabinoid. The methods provided herein may comprise one or more of (1) an extraction step wherein cannabinoid compounds are extracted from Cannabis plant material, thereby producing a composition comprising THCV; (2) a distillation step wherein a composition comprising THCV is distilled, thereby providing a distillate that is enriched in THCV; and (3) a flash chromatography step wherein a composition comprising THCV is subjected to flash chromatography, thereby producing a THCV isolate.

### CROSS-REFERENCE TO RELATED APPLICATIONS

This application claims priority to U.S. Provisional Application No. 63/347,542, filed May 31, 2022, the entire contents of which are incorporated herein by reference.

### BACKGROUND

Cannabinoids are a family of chemical compounds derived from the cannabis plant. For example, tetrahydrocannabinol (THC) is known as the principal psychoactive constituent of cannabis.

Δ⁹-Tetrahydrocannabivarin (THCV) is a minor cannabinoid and a chemical analog of Δ⁹-tetrahydrocannabinol. THCV is found in significative amounts only in high THC cannabis strains. Unlike THC, however, THCV is not listed as Schedule I drug by the US federal government. Human studies have shown that THCV is only about 25% as psychoactive as THC. It has a quicker onset of action than THC and is typically of briefer duration.

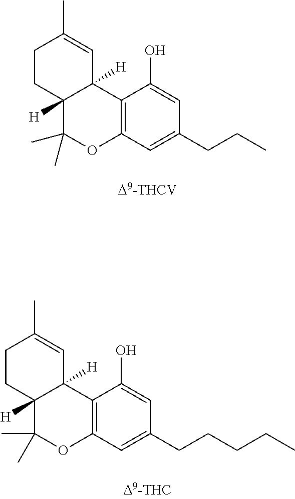

Recent research has identified several promising therapeutic applications of THCV. For example, it appears to reduce appetite in mice models, and to reduce certain markers of Type 2 diabetes in humans. THCV has also shown promising results in rodent models for the treatment of Parkinson's disease and epilepsy.

Due to these and other potential therapeutic benefits, there is an increasing interest in methods for the separation and isolation of THCV. Currently, the primary challenge is the high cost of THCV production. There is therefore a need in the art for efficient, cost-effective methods of separating, enriching, and/or isolating THCV.

### SUMMARY

Provided herein are methods for the isolation of THCV from a composition comprising THCV and at least one other cannabinoid. The methods provided herein may comprise one or more of (1) an extraction step wherein cannabinoid compounds are extracted from Cannabis plant material, thereby producing a composition comprising THCV; (2) a distillation step wherein a composition comprising THCV is distilled, thereby providing a distillate that is enriched in THCV; and (3) a flash chromatography step wherein a composition comprising THCV is subjected to flash chromatography, thereby producing a THCV isolate.

For example, provided herein is a method for isolating THCV from a starting material, the method comprising a distillation step wherein a starting material comprising THCV is distilled, thereby providing a distillate that is enriched in THCV relative to the starting material; and a flash chromatography step wherein the distillate is subjected to flash chromatography, thereby producing a THCV isolate composition.

Also provided herein is a method for isolating THCV from a starting material comprising THC, the method comprising a distillation step wherein the starting material is distilled using a wiped thin-film evaporator at an internal condenser temperature of from about 70° C. to about 110° C. and a pressure of from about 140 mTorr to about 220 mTorr, thereby providing a distillate that is enriched in THCV relative to the starting material.

Also provided herein is a cannabinoid composition comprising THCV, wherein the composition is produced by a method as provided herein.

Other objects and features will be in part apparent and in part pointed out hereinafter.

### BRIEF DESCRIPTION OF THE DRAWINGS

FIG. 1 is a chart depicting the THCV concentration (percentage by weight, left bar) and the THC concentration (percentage by weight, right bar) in distillate before and after distillation at 120° C. heating mantel temperature (“HMT”) and 180 mTorr pressure, as described in further detail in Example 2.

FIG. 2 is graph depicting the separation of THCV from THC using a mix of ethanol and water (65:35) in a Selekt Biotage flash chromatography system equipped with a Biotage Sfär C18 400 g column (CV=582 mL) and a UV detector (230 nm), as described in further detail in Example 3. As used herein the abbreviation “CV” refers to the dead volume of the chromatography column.

FIG. 3 is an HPLC chromatogram of THCV using an Agilent 1100 HPLC system equipped with a Phenomenex Kinetex C18 2.6 μm C18 100A (150×4.6 mm) analytical column and a UV detector (228 nm) and using an isocratic mobile phase acetonitrile/water phosphoric acid 0.1% (75:25) and a flow of 1.0 ml/min following distillation (Example 2, FIG. 1) and C18 reversed phase flash chromatography (Example 3, FIG. 2), as described in further detail in Example 4.

FIG. 4 is a graph depicting partial THCV/THC separation using a gradient n-heptane/acetone (acetone 0 to 15%) in a Selekt Biotage flash chromatography system equipped with a Biotage Sfar HC Duo (normal silica) 10 g column (CV=15 mL), as described in further detail in Example 5.

FIG. 5 is a graph depicting the THCVA/THCA ratio following the distillation of the “Orange” extract at 135° C. HMT/84° C. ICT 135° C. and HMT/84° C. ICT, as described in further detail in Example 6. “Oil” stands for cold ethanol extract.

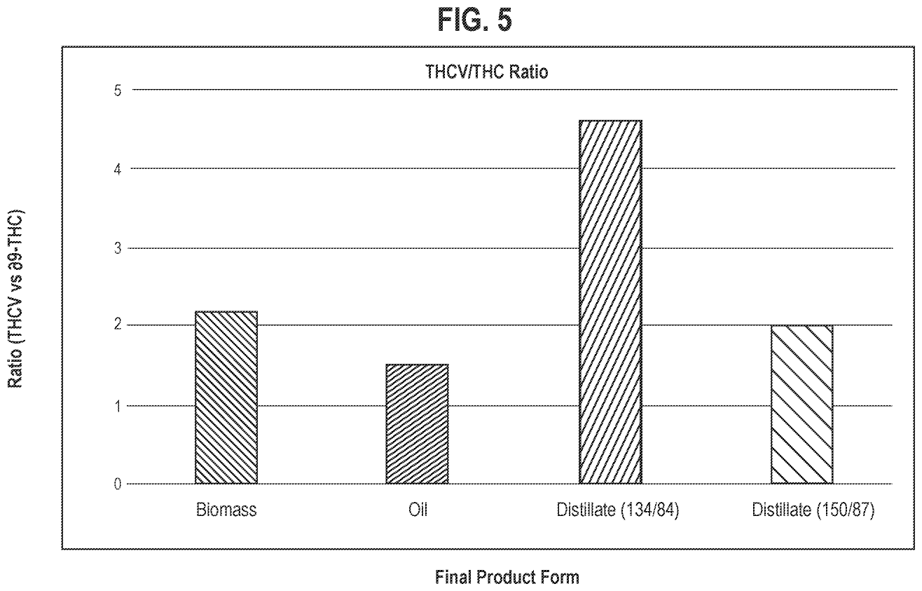

### DETAILED DESCRIPTION

Provided herein are methods for the isolation of naturally occurring THCV from a cannabinoid-containing starting material (e.g., from Cannabis plant material or an extract thereof). The provided methods are useful, for example, to efficiently and cost-effectively separate THCV from THC (and optionally, other cannabinoids) that may be present in the starting material.

The methods provided herein may utilize distillation, flash chromatography, or a combination thereof to obtain isolated THCV. For example, it has been discovered that distillation under certain conditions (e.g., temperature and pressure) allows for the partial separation of THCV from THC, thereby providing a distillate having an enriched THCV content relative to a starting material comprising a mixture of THCV and THC. It has additionally been discovered that THCV can be further isolated by flash chromatography, for example, using a preparative reversed silica C18 column and a mixture of ethanol/water as the eluant.

These and other exemplary features of the provided methods are described in further detail below.

### Definitions

As used herein, THC refers to tetrahydrocannabinol, and is inclusive of isomers including but not limited to Δ⁸-THC, Δ⁹-THC, Δ¹⁰-THC, and exo-THC.

As used herein, THCV refers to tetrahydrocannabivarin, and is inclusive of isomers including but not limited to Δ⁸-THCV, Δ⁹-THCV, Δ¹⁰-THCV, and exo-THCV.

As used herein, THCA refers to tetrahydrocannabinolic acid, and is inclusive of isomers including but not limited to Δ⁸-THCA, Δ⁹-THCA, Δ¹⁰-THCA, and exo-THCA.

As used herein, THCVA refers to tetrahydrocannabivarinic acid, and is inclusive of isomers including but not limited to Δ⁸-THCVA, Δ⁹-THCVA, Δ¹⁰-THCVA, and exo-THCVA.

### Reaction Procedure

The methods provided herein may comprise one or more of (1) an extraction step wherein cannabinoid compounds are extracted from Cannabis plant material, thereby producing a cannabis extract composition comprising THCV; (2) a distillation step wherein a distillation starting material comprising THCV is distilled, thereby providing a distillate that is enriched in THCV relative to the starting material; and (3) a flash chromatography step wherein a composition comprising THCV is subjected to flash chromatography, thereby producing a THCV isolate composition. These steps are described in further detail below.

### Extraction Step

The methods provided herein may comprise an extraction step wherein cannabinoid compounds are extracted from Cannabis plant material, thereby producing a composition comprising THCV.

The extraction step may comprise drying the Cannabis plant material. For example, the Cannabis plant material may be dried for a period of at least about 6 hours, at least about 12 hours, at least about 1 day, at least about 2 days, at least about 3 days, at least about 4 days, at least about 5 days, at least about 6 days, or at least about 7 days or more.

The Cannabis plant material may optionally be dried at an elevated temperature (i.e., a temperature greater than about 25° C.). For example, the Cannabis plant material may be dried at a temperature of at least about 30° C., at least about 35° C., at least about 40° C., at least about 45° C., or at least about 50° C.

Optionally, the Cannabis plant material may be dried under a vacuum (e.g., at a pressure of less than about 30 kPa).

The extraction step may comprise freezing the Cannabis plant material. For example, the Cannabis plant material may be frozen for a period of at least about 6 hours, at least about 12 hours, at least about 1 day, at least about 2 days, at least about 3 days, at least about 4 days, at least about 5 days, at least about 6 days, or at least about 7 days or more. The Cannabis plant material may be frozen at a temperature of less than about 0° C., for example, less than about −10° C. or less than about −20° C.

The extraction step may comprise grinding the Cannabis plant material. The Cannabis plant material may optionally be frozen, dried, or both (i.e., frozen and dried) prior to grinding.

The extraction step may comprise contacting the Cannabis plant material with a solvent, thereby forming a cannabis extract composition. Non-limiting examples of solvents include methanol, ethanol, isopropanol, acetonitrile, and ethyl acetate. A preferred solvent is ethanol. The contacting step may be conducted at a reduced temperature (i.e., a temperature less than about 25° C.). For example, the contacting step may be conducted at a temperature of less than about 20° C., less than about 10° C., less than about 5° C., or less than about 0° C.

The extraction step may comprise decarboxylation of the cannabis extract composition. The decarboxylation may be carried out at a temperature, for example, of from about 110° C. to about 160° C. For example, the cannabis extract composition may be decarboxylated at a temperature of at least about 110° C., at least about 120° C., at least about 130° C., at least about 140° C., at least about 150° C., or at least about 160° C. for a period of at least about 30 minutes, at least about 1 hour, at least about 2 hours, at least about 4 hours, at least about 6 hours, at least about 8 hours, or at least about 12 hours.

Optionally, the cannabis extract composition may be decarboxylated under a vacuum (e.g., at a pressure of less than about 100 Torr, for example from about 50 Torr to about Torr).

Distillation Step

The methods provided herein may comprise a distillation step wherein a distillation starting material comprising THCV is distilled, thereby providing a distillate that is enriched in THCV. The distillation starting material may be, for example, a cannabis extract composition that is produced as described above.

As a non-limiting example, the distillation step may be conducted using a wiped film evaporator, also referred to as a thin film evaporator or a wiped thin-film evaporator.

The distillation step may be conducted at a heating mantel temperature of about 135° C. For example, the distillation step may be conducted at a heating mantel temperature of from about 100° C. to about 160° C. As non-limiting examples, the distillation step may be conducted at a heating mantel temperature of at least about 100° C., at least about 110° C., at least about 120° C., or at least about 130° C. The distillation step may be conducted at a heating mantel temperature of no higher than about 160° C., no higher than about 150° C., or no higher than about 140° C.

The distillation step may be conducted at an internal condenser temperature of about 90° C. For example, the distillation step may be conducted at an internal condenser temperature of from about 70° C. to about 110° C. As non-limiting examples, the distillation step may be conducted at an internal condenser temperature of at least about 70° C., at least about at least about 80° C., or at least about 85° C. The distillation step may be conducted at an internal condenser temperature of no higher than about 110° C., no higher than about 105° C., no higher than about 100° C., or no higher than about 95° C.

The distillation step may be conducted under a vacuum (e.g., at a pressure of about 1000 mTorr or less). Without being bound to a particular theory, lower distillation pressures are preferred because they lower the boiling point of the THCV component and allow for a better separation of THCV from the other components present in the starting material. For example, the distillation step may be conducted at a pressure of less than about 1000 mTorr, less than about 800 mTorr, less than about 600 mTorr, less than about 500 mTorr, less than about 420 mTorr, less than about 400 mTorr, less than about 350 mTorr, or less than about 300 mTorr. In some embodiments, the distillation step may be carried out at a very low pressure of less than about 250 mTorr, less than about 200 mTorr, less than about 150 mTorr, or even less than about 100 mTorr. As a non-limiting example, the distillation step may be conducted at a pressure of from about 140 mTorr to about 220 mTorr (e.g., at a pressure of about 180 mTorr).

The methods provided herein may comprise two or more distillation steps. For example, it may be desirable to undertake one or more distillation steps at temperatures below the ranges generally described above in order to remove volatile components having a low boiling point (e.g., terpenes).

The distillation step provides a distillate that is enriched in THCV, relative to the concentration of THCV in the distillation starting material. In general, the concentration of THCV in the distillate will depend on the initial concentration of THCV in the starting material prior to the distillation step.

For example, the distillate may comprise THCV in a concentration of at least about 20% by weight, at least about 25% by weight, at least about 30% by weight, at least about 40% by weight, at least about 45% by weight, or at least about 50% by weight.

In preferred embodiments, THCV comprises a significant portion of the total cannabinoid content of the distillate. For example, THCV may comprise at least about 20%, at least about 25%, at least about 30%, at least about 35%, at least about 40%, at least about 45%, at least about 50%, at least about 55%, or at least about 60% of the total cannabinoid content of the distillate.

The distillate may comprise a ratio of THCV to THC of at least about 0.5:1, at least about 0.6:1, at least about 0.7:1, at least about 0.8:1, at least about 0.9:1, or at least about 1:1 on a weight basis. In preferred embodiments, the distillate comprises THCV in excess relative to THC on a weight basis.

The distillation step may provide a distillate having a ratio of THCV to THC that exceeds the ratio of THCV to THC in the starting material by a factor of at least about 1.5, and preferably a factor of at least about 2. For example, for a starting material having an approximate 1:1 ratio of THCV to THC, the distillate may comprise a ratio of THCV to THC of at least about 1.5:1, at least about 1.75:1, or at least about 2:1. For a starting material having an approximate 2:1 ratio of THCV to THC, the distillate may comprise a ratio of THCV to THC of at least about 3:1, at least about 3.5:1, or at least about 4:1. For a starting material having an approximate 3:1 ratio of THCV to THC, the distillate may comprise a ratio of THCV to THC of at least about 4:1, at least about 5:1, or at least about 6:1.

Flash Chromatography Step

The methods provided herein may comprise a flash chromatography step wherein a composition comprising THCV is subjected to flash chromatography, thereby producing a THCV isolate composition. The composition comprising THCV may be, for example, a distillate that is enriched in THCV and produced using a distillation step as described above.

As a non-limiting example, the flash chromatography step may comprise a reversed-phase chromatography step. The reversed-phased chromatography step may be conducted using, for example, a reversed-phase silica C18 chromatography column.

The flash chromatography step may be conducted using an eluent comprising a mixture of water and a solvent. The solvent may be selected from the group consisting of alcohols, ethyl acetate, and acetonitrile. For example, the flash chromatography step may be conducted using an eluent comprising a mixture of water and alcohol. Non-limiting examples of alcohols include methanol, ethanol, isopropanol, and other aliphatic organic alcohols. Preferably, the alcohol is ethanol.

The eluent may comprise the alcohol in a concentration of at least about 50% by weight. For example, the eluent may comprise the alcohol in a concentration of about 65% by weight (i.e., a 65:35 ratio of alcohol to water). For example, the eluent may comprise the alcohol in a concentration of from about 50% to about 10% by weight. As non-limiting examples, the eluent may comprise an alcohol concentration of at least about 50% by weight, at least about 55% by weight, or at least about 60% by weight.

THCV Isolate Composition

The flash chromatography step provides a THCV isolate composition.

The THCV isolate composition may comprise THCV in a concentration of at least about 50%, at least about 60%, at least about 70%, at least about 75%, at least about 80%, at least about 85%, at least about 90%, at least about 95%, at least about 96%, at least about 97%, at least about 98%, or at least about 99% by weight, relative to the total weight of the composition.

THCV comprises a majority of the total cannabinoid content of the THCV isolate. For example, THCV may comprise at least about 50%, at least about 60%, at least about 70%, at least about 75%, at least about 80%, at least about 85%, at least about 90%, at least about 95%, at least about 96%, at least about 97%, at least about 98%, or at least about 99% of the total cannabinoid content of the THCV isolate composition.

The THCV isolate composition may have a boiling point (at standard atmospheric pressure) that is greater than the boiling point of THC. For example, the THCV isolate composition may have a boiling point (at standard atmospheric pressure) of greater than about 200° C., greater than about 210° C., or greater than about 220° C. Without being bound to a particular theory, it has been surprisingly discovered that the boiling point of THCV is greater than that of THC at standard atmospheric pressure, yet at reduced pressure and a specific heating mantel temperature, THCV is enriched by distillation.

### Cannabinoid Compositions

Also provided herein is a cannabinoid composition comprising THCV, wherein the composition is produced by a method as described above.

The cannabinoid composition may comprise THCV in any of the amounts, concentrations, or ratios as described above. For example, the cannabinoid composition may comprise a THCV isolate composition as described above.

### EXAMPLES

The following non-limiting examples are provided to further illustrate the present disclosure.

### Example 1

A high Δ⁹-tetrahydrocannabivarin acid (THCVA) cannabis strain was grown for 8 weeks and then harvested. 14,210 grams of harvested material were dried for seven days and ground, producing 2,100 grams of dried ground plant material. This material was then frozen for 3 days and extracted using a Cup-15 cold ethanol extractor (Delta Separations, Cotati, CA). The ethanol was evaporated using a Falling Film Evaporator (Delta Separations) and the resulting extract was treated at 110° C. under vacuum for 12 hours for the decarboxylation of cannabinoids and elimination of residual ethanol using a 50 L glass reactor under vacuum system from Keda Instruments (Zhengzhou, China). The weight of the extract was 302.5 grams containing 21.6% THCV and 46.7% THC.

### Example 2

A cannabinoid extract, prepared as described in Example 1, was subjected to distillation as described below.

The distillation system was a 6 inches stainless-steel wiped-film evaporator system from Pope Scientific (Saukville, Wisconsin). As used in this example, “HMT” refers to the heating mantel temperature of the system and “ICT” refers to the internal condenser temperature of the system.

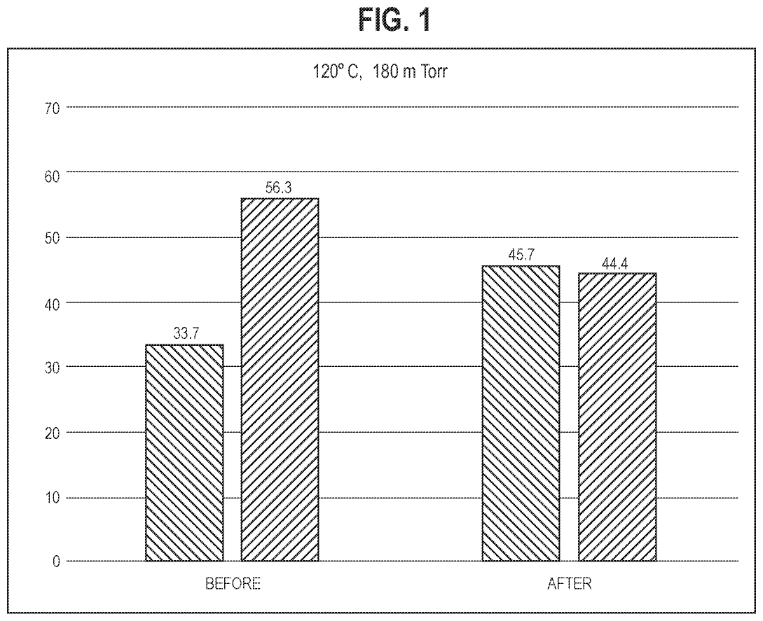

Two passes were conducted at low temperature (115° C. HMT and 42° C. ICT) and low vacuum (5 Torr and 2 Torr pressures, respectively) to eliminate terpenes and other undesired volatiles (approximately 14 grams). A third pass was conducted at 165° C. HMT and 430 mTorr pressure resulting in 156.8 grams of distillate containing 28.7% THCV and 48.1% THC, and grams of distillate waste (dark matter) containing 2.8% THCV and 12.4% THC. A fourth pass at 140° C. HMT and 180 mTorr pressure resulted in the distillation of both THCV and THC with a distillate containing 33.7% THCV and 56.3% THC, therefore with no significant separation. A fifth and final pass at 120° C. HMT and 180 mTorr pressure resulted in 29.9 grams distillate (yellow oil) containing 45.7% THCV and 44.4% THC, therefore going from a 1:2 THCV/THC ratio to a 1:1 ratio (see Table 1 and FIG. 1), while the “waste” fraction (non-evaporated) contained 28.1% THCV and 58.6% THC.

TABLE 1
Wiped-film evaporator pass conditions (HMT, ICT, and pressure),
distillate amount obtained (g) and THCV and THC content (%).

| | HMT | ICT | Pressure | Dist. (g) | % THCV | % THC |
|---|---|---|---|---|---|---|
| Extract | | | | 302.5 | 21.6 | 46.7 |
| Pass 1 | 115° C. | 42° C. | 5 Torr | | 24.1 | 48.8 |
| Pass 2 | 115° C. | 42° C. | 2 Torr | | | |
| Pass 3 | 165° C. | 80° C. | 430 mTorr | 156.8 | 28.7 | 48.1 |
| Pass 4 | 140° C. | 95° C. | 180 mTorr | 130.0 | 33.7 | 56.3 |
| Pass 5 | 120° C. | 90° C. | 180 mTorr | 29.9 | 45.7 | 44.4 |

Without being bound to a particular theory, these results indicate that the boiling point of THCV is different enough from the boiling point of THC to allow enriching THCV from a mix of THCV/THC. At atmospheric pressure, the boiling point of THCV is higher than THC. At low pressure, however, distillation using specific conditions of temperature, i.e., about 120° C. for the heating mantel temperature (HMT) and about 90° C. for the internal condenser temperature (ICT), and about 180 mTorr for the pressure, allowed to cut by half the amount of THC compared to THCV (see FIG. 1). Therefore, THCV can partially be separated from THC by distillation under the low pressure conditions described herein. The total amount of THC+THCV measured in the distillate obtained in these conditions was 90% w/w, which is consistent with distillate obtained with high THC strains. The other 10% are identified and unidentified plant molecules such as minor cannabinoids, terpenes, flavonoids, and waxes with similar boiling points (115-170° C. range). THCV enrichment by distillation therefore allows for significant reductions in the cost of subsequent THCV isolation by flash chromatography.

### Example 3

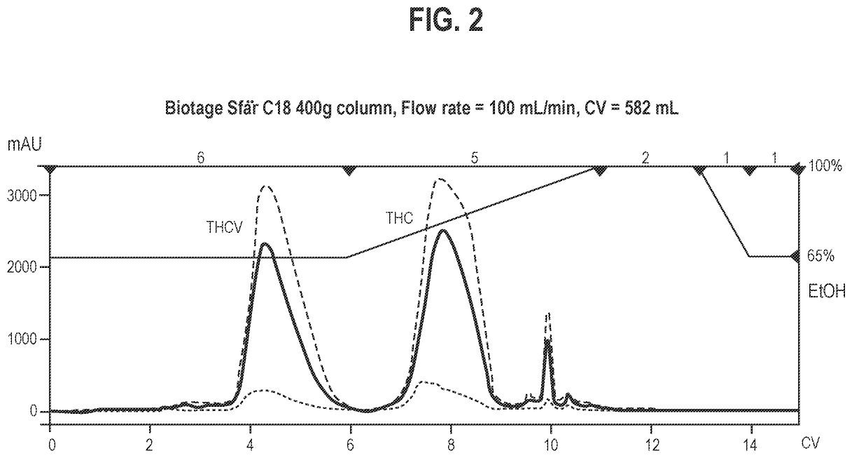

A method was developed to separate THCV from THC using a mix of water/ethanol (35:65) at a flow of 100 ml/min in a Biotage Sfar C18 400 g column with a Selekt Biotage flash chromatography system (FIG. 2). 4 grams of distillate were dissolved in 20 ml of ethanol, and 5 ml of water were added prior to injection. Adding more than 5 mL water can results in the precipitation of the cannabinoids. Approximately 1 L of the THCV fraction was collected starting at approximately 4 CV (24 minutes) after injection. The ethanol and water were evaporated with a rotary evaporator, leading to 1.3 grams of a yellow oil.

Collected fractions were analyzed by HPLC, as described in Example 5 below. Seven runs were conducted, leading to 9.2 grams of 95% THCV.

### Example 4

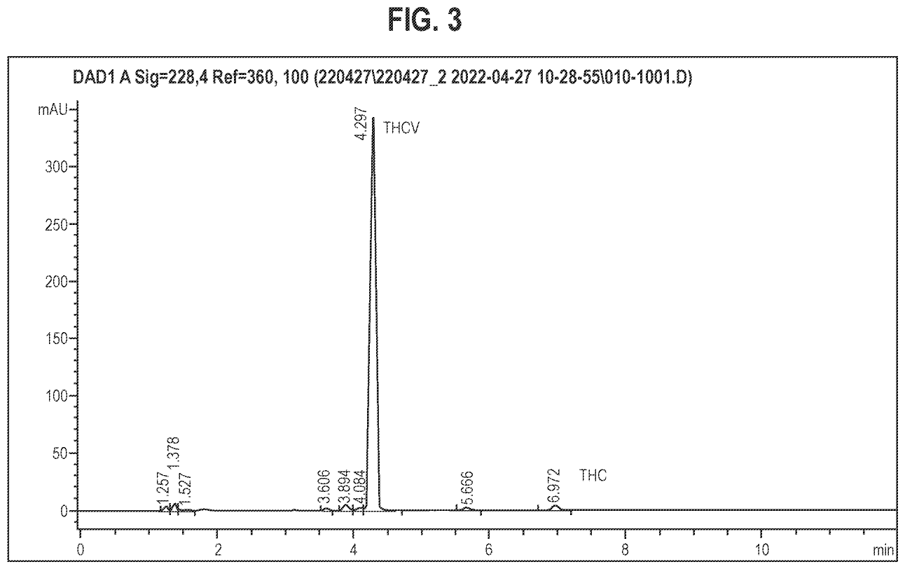

THCV and THC levels in cannabis extract and distillate were monitored using an Agilent 1100 HPLC system equipped with a Phenomenex Kinetex 2.6 μm C18 100A (150×4.6 mm) column (Torrance, CA) and an isocratic mobile phase of a mix acetonitrile/water with 0.1% phosphoric acid (75:25), and a flow of 1.0 ml/min (12 minutes total run); the retention times of THCV and THC were 4.2 and 7.0 minutes, respectively (FIG. 3). THCV and THC were also analyzed by GC-FID using a Restek Rxi-5Sil-MS 0.25 mmID 0.25 μm df (30 meters) column, hydrogen as carrier gas, and a temperature ramp of 10° C./min from 50° C. (1 min hold) to 300° C. (27 minutes total run); the retention times of THCV and THC were 21.98 and 23.62 minutes, respectively. Samples were dissolved in ethanol and filtered prior to analysis. THCV and THC standards were obtained from Cerilliant (Round Rock, Texas).

These preliminary results show that THCV can partially be separated from THC by distillation using a lower distillation temperature, i.e., 120° C. vs. 140° C. at 180 mTorr, and further isolated by flash chromatography using a C18 column and a mix ethanol/water (65:35) as eluant. Additional experiments are needed to determine the precise boiling point of THCV and the full optimal conditions for its separation from THC by distillation, i.e., heating mantel temperature (HMT), internal condenser temperature (ICT) and the pressure in the system. Indeed, while the HMT and the pressure are the driving parameters for an effective distillation, the optimal ICT can significantly improve the effectiveness by increasing the selectivity of the condensation. Note that distillation prior to flash chromatography, by eliminating the dark matter waste, also allows more re-uses of costly preparative columns. To conclude, distillation followed by C18 flash chromatography is a cost-effective method for the preparation of pure THCV.

### Example 5

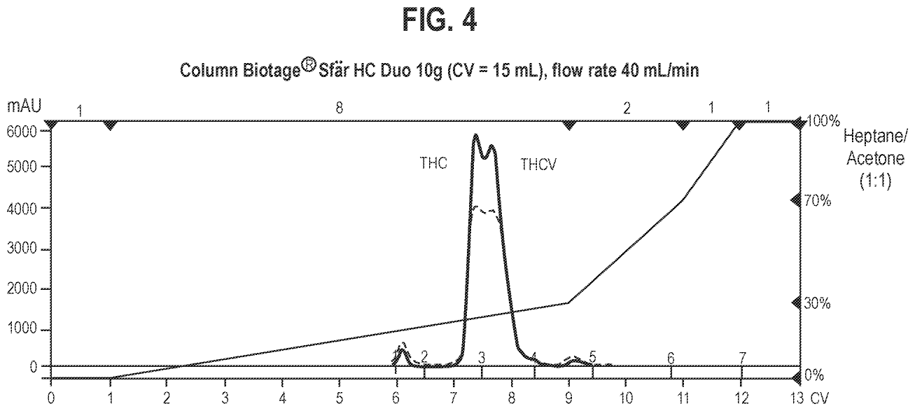

Partial separation of THCV from THC was also obtained using methods similar to those described above in Example 3 above, but with a gradient of n-heptane/acetone (acetone from 0 to 15%) using a Biotage Sfar HC Duo 10 g (normal silica). The results of this separation procedure are depicted in FIG. 4.

### Example 6

Three different extracts (Green, Orange, Red) were run through the Pope 6″ stainless steel wiped film distillation equipment. Between 3 and 3.5 Kgs of extract were put into the receiving container. Note that a first pass at low HMT to eliminate terpenes was not performed. Feeding speed, blade rotation speed, pressure, heat mantle temperature (aka HMT, HM, or external heat bands) and the internal condenser temperature (aka ICT or IC) were set (see parameters above) and extract processed. Every half-hour, the system was depressurized, the weight of both distillate and waste oil containers measured and testing samples of both distillate (between 30-300 g) and waste oil (between 50-250 g) collected. The HMT and/or ICT parameters were changed, and the process resumed. As a result, 10 to 12 different parameters varying HMT and ICT were run per extract.

The “Green” and “Orange” extracts were approximately 1 part THCA and 3-parts THCVA in flower form, or 1:3, whereas the “Red” extract was approximately 1 part THCA and 1-part THCVA in flower form, or 1:1. The extracts were tested using the conditions set forth below in Table 2, where “HM° C.” refers to the heating mantel temperature in Celsius, and “IC° C.” refers to the internal condenser temperature in Celsius.

TABLE 2
Test Conditions for Green, Orange, and Red Extracts

| Green Extract | | Orange Extract | | Red Extract | |
|---|---|---|---|---|---|
| 3142 g | | 3307 g | | 3534 g | |
| HM° C. | IC° C. | HM° C. | IC° C. | HM° C. | IC° C. |
| 120 | 80 | 135 | 80 | 135 | 80 |
| | 85 | | 84 | | 84 |
| | 90 | | 87 | | 87 |
| 135 | 80 | 142 | 80 | 142 | |
| | 85 | | 84 | | 84 |
| | 90 | | 87 | | 87 |
| 150 | 80 | 150 | 84 | 150 | 84 |
| | 85 | | 87 | | 87 |
| | 90 | | 90 | | 90 |
| 165 | 80 | | | 157 | 84 |
| | | | | | 87 |
| | | | | | 90 |
| | | | | 164 | 84 |
| | | | | | 87 |
| | | | | | 90 |

The average obtained THCV/THC ratio, potency, and yield as a function of the ICT, at a constant HMT of 135° C., are presented below in Table 3. Corresponding values taken at a constant HMT of 150° C., are presented below in Table 4. As used in the following tables, “potency” refers to the combined weight percentage of THCV and THC.

TABLE 3
Average THCV/THC Ratio, Potency, and Yield at Constant HMT of 135° C.

| Ratio | | | | Potency | | | | Yield | | | |
|---|---|---|---|---|---|---|---|---|---|---|---|
| IC° C. | Red | Orange | Green | IC° C. | Red | Orange | Green | IC° C. | Red | Orange | Green |
| 80 | 1.4 | 3.0 | 4.5 | 80 | 88.2 | 80.5 | 81.1 | 80 | 23.3 | 16.9 | 32.4 |
| 84 | 1.6 | 4.6 | 5.1 | 84 | 86.1 | 87.6 | 89.6 | 84 | 30.4 | 16.9 | 15.6 |
| 87 | 1.5 | 3.8 | | 87 | 87.0 | 88.4 | | 87 | 20.5 | 21.6 | |
| 90 | | | 5.0 | 90 | | | 100.2 | 90 | | | 18.1 |

TABLE 4
Average THCV/THC Ratio, Potency, and Yield at Constant HMT of 150° C.

| Ratio | | | | Potency | | | | Yield | | | |
|---|---|---|---|---|---|---|---|---|---|---|---|
| IC° C. | Red | Orange | Green | IC° C. | Red | Orange | Green | IC° C. | Red | Orange | Green |
| 80 | 1.1 | 1.9 | 2.4 | 80 | 92.0 | 91.9 | 98.9 | 80 | 66.5 | 78.1 | 74.5 |
| 84 | 0.9 | 2.0 | 2.6 | 84 | 91.0 | 91.8 | 90.7 | 84 | 73.9 | 87.5 | 86.4 |
| 87 | 1.0 | 2.0 | | 87 | 88.5 | 95.6 | | 87 | 73.9 | 80.4 | |
| 90 | | | 2.2 | 90 | | | 85.8 | 90 | | | 81.3 |

A summary of the THCV/THC ratio at harvest, extraction, and distillation for the Orange cultivar is presented in Table 5 below. These results are presented graphically in FIG. 6.

TABLE 5
Orange Distillate Summary

| | | | Distillation (HMT/ICT) | | |
|---|---|---|---|---|---|
| | Harvest* | Extraction | 135/84 | 135/87 | 150/87 |
| THCV (%) | 19.7 | 48.5 | 71.9 | 70.1 | 61 |
| THC (%) | 9.2 | 31.8 | 15.7 | 18.3 | 30.8 |
| Ratio | 2.15 | 1.53 | 4.6 | 3.8 | 2 |
| Weight (g) | 22831 | 3307 | 43 | 51 | 337 |
| Yield (%) | | | 16.9 | 21.6 | 87.5 |

*Values in the Harvest column are for THCVA (rather than THCV) and THCA (rather than THC).

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

In view of the above, it will be seen that the several objects of the disclosure are achieved and other advantageous results attained.

As various changes could be made in the above products and methods without departing from the scope of the disclosure, it is intended that all matter contained in the above description shall be interpreted as illustrative and not in a limiting sense.

What is claimed is:

1. A method for isolating THCV from a starting material, the method comprising:
a distillation step wherein a starting material comprising THCV is distilled, thereby providing a distillate that is enriched in THCV relative to the starting material; and
a flash chromatography step wherein the distillate is subjected to flash chromatography, thereby producing a THCV isolate composition.

2. The method of claim 1 wherein the distillation step is conducted using a wiped film evaporator.

3. The method of claim 2 wherein the distillation step is conducted at a heating mantel temperature of from about 100° C. to about 160° C.

4. The method of claim 3 wherein the distillation step is conducted at a heating mantel temperature of about 135° C.

5. The method of claim 2 wherein the distillation step is conducted at an internal condenser temperature of from about 70° C. to about 110° C.

6. The method of claim 5 wherein the distillation step is conducted at an internal condenser temperature of about 85° C.

7. The method of claim 1 wherein the distillation step is conducted at a pressure of less than about 1000 mTorr.

8. The method of claim 7 wherein the distillation step is conducted at a pressure of less than about 420 mTorr.

9. The method of claim 1 wherein the flash chromatography step comprises a reversed-phase chromatography step.

10. The method of claim 1 wherein the flash chromatography step is conducted using an eluent comprising a mixture of water and alcohol.

11. The method of claim 10 wherein the eluent comprises a mixture of water and ethanol.

12. The method of claim 11 wherein the eluent comprises ethanol in a concentration of at least about 60% by weight.

13. The method of claim 1 wherein the THCV isolate composition comprises THCV in a concentration of at least about 90% by weight, relative to the total weight of the composition.

14. The method of claim 1 wherein THCV comprises at least about 90% of the total cannabinoid content of the THCV isolate composition.

15. A cannabinoid composition comprising a THCV isolate produced by the method of claim 1.

16. A method for isolating THCV from a starting material comprising THC, the method comprising:
a distillation step wherein the starting material is distilled using a wiped film evaporator at an internal condenser temperature of from about 80° C. to about 90° C. and a pressure of less than about 1000 mTorr,
thereby providing a distillate that is enriched in THCV relative to the starting material.

17. The method of claim 16 wherein the distillation step is conducted at a heating mantel temperature of from about 110° C. to about 150° C.

18. The method of claim 16 wherein the distillate comprises THCV in a concentration of at least about 40% by weight.

19. The method of claim 16 wherein the distillate comprises THCV in excess relative to THC on a weight basis.

20. A cannabinoid composition comprising a distillate produced by the method of claim 16.
